(19) United States
(12) Patent Application Publication
(10) Pub. No.: US 2026/0000417 A1
(43) Pub. Date: Jan. 1, 2026

(54) ASPIRATION CATHETER WITH REINFORCED DISTAL TIP MEMBER

(71) Applicant: BOSTON SCIENTIFIC SCIMED, INC., Maple Grove, MN (US)

(72) Inventors: Robert D. Cooper, Andover, MN (US); Alyssa Madej, Minnetonka, MN (US)

(73) Assignee: BOSTON SCIENTIFIC SCIMED, INC., Maple Grove, MN (US)

(21) Appl. No.: 19/247,368

(22) Filed: Jun. 24, 2025

Related U.S. Application Data
(60) Provisional application No. 63/665,321, filed on Jun. 28, 2024.

(51) Int. Cl. A61B 17/22; A61M 25/00

(52) U.S. Cl. CPC A61B 17/22; A61M 25/0026; A61M 25/0071; A61B 2017/22038; A61B 2017/22079; A61B 2217/005; A61M 2025/0039

(57) ABSTRACT

An aspiration catheter for use in a thrombectomy procedure is disclosed. The aspiration catheter includes a guidewire shaft having a proximal end region, a distal end region and a guidewire lumen extending within at least a portion of a distal tip member. The distal tip member is coupled to the distal end region of the catheter shaft and includes a plurality of spokes extending between the guidewire shaft and an inner surface of the distal tip member.

### CROSS REFERENCE TO RELATED APPLICATIONS

The present application claims the benefit of U.S. Provisional Patent Application Ser. No. 63/665,321, filed Jun. 28, 2024, the disclosure of which is incorporated herein by reference.

### TECHNICAL FIELD

The disclosure is directed to aspiration systems. More particularly, the disclosure is directed to an aspiration catheter having a reinforced distal tip member coupled to the aspiration catheter distal end.

### BACKGROUND

Thrombectomy is a procedure for removing thrombus from the vasculature of a patient. Mechanical and fluid-based systems can be used to remove thrombus. With fluid-based systems, an infusion fluid may be infused to a treatment area of a vessel with a catheter to dislodge the thrombus. In some instances, an effluent (e.g., the infusion fluid and/or blood) including the dislodged thrombus may be aspirated from the vessel through the catheter. Of the known thrombectomy systems and methods, there is an ongoing need to provide alternative configurations of thrombectomy catheters, aspiration catheters and systems, as well as methods of operating such thrombectomy systems.

### SUMMARY

This disclosure provides design, material, manufacturing method, and use alternatives for medical devices. An example aspiration catheter includes a catheter shaft having a proximal end region, a distal end region and a lumen extending therein. Example aspiration catheters may also include a distal tip member coupled to the distal end region of the catheter shaft and may further include a guidewire shaft having a proximal end region, a distal end region and a guidewire lumen extending therein. The guidewire shaft may extend within at least a portion of the lumen of the catheter shaft and within the distal tip member. The distal tip member may include a plurality of spokes extending between the guidewire shaft and an inner surface of the distal tip member.

Alternatively or additionally to any of the examples above, the distal tip member may further include a hub with an aperture therein, and the guidewire shaft may extend through the aperture of the hub.

Alternatively or additionally to any of the examples above, the aperture of the hub may be centrally located along a central longitudinal axis of the distal tip member, such that the plurality of spokes maintain the guidewire lumen substantially coaxial with the central longitudinal axis of the distal tip member.

Alternatively or additionally to any of the examples above, the plurality of spokes may define a plurality of passages between the plurality of spokes, the plurality of passages may allow passage of entrained fluid into the lumen of the catheter shaft.

Alternatively or additionally to any of the examples above, a distal portion of the distal tip member may be tapered at an angle of at least 15 degrees with respect to a central longitudinal axis of the distal tip member.

Alternatively or additionally to any of the examples above, the catheter shaft may further include a fluid jet loop disposed therein. The fluid jet loop including a fluid jet loop lumen with a plurality of jet orifices extending through a wall of the fluid jet loop.

Alternatively or additionally to any of the examples above, the fluid jet loop may encircle at least a portion of the guidewire lumen such that the plurality of jet orifices are oriented in a proximal direction.

Alternatively or additionally to any of the examples above, the fluid jet loop may include one or more distally facing jet orifices oriented toward the distal tip member.

Alternatively or additionally to any of the examples above, the one or more distally facing jet orifices may be directed toward the plurality of passages of the distal tip member.

Another example aspiration catheter includes a catheter shaft having a proximal end region, a distal end region, a lumen extending therein and a central longitudinal axis. In this and other examples, the aspiration catheter may further include a distal tip member coupled to the distal end region of the catheter shaft. The aspiration catheter of this and other examples may further include a guidewire shaft having a proximal end region, a distal end region and a guidewire lumen extending therein. The guidewire shaft may extend within at least a portion of the lumen of the catheter shaft and within the distal tip member. The distal tip member may include a plurality of spokes radially arranged around the guidewire shaft to support the guidewire shaft such that the guidewire shaft is coaxial with the central longitudinal axis.

Alternatively or additionally to any of the examples above, the plurality of spokes may define one or more passages therebetween. The plurality of passages may allow passage of entrained fluid into the lumen of the catheter shaft.

Alternatively or additionally to any of the examples above, a distal portion of the distal tip member is tapered at an angle of at least 15 degrees with respect to the central longitudinal axis.

Alternatively or additionally to any of the examples above, the catheter shaft may further include a fluid jet loop disposed therein. The fluid jet loop may include a fluid jet loop lumen with a plurality of jet orifices extending through a wall of the fluid jet loop, and the fluid jet loop may further encircle the guidewire shaft.

Another example aspiration catheter includes a thrombectomy catheter including a catheter shaft having a proximal end region, a distal end region and a lumen extending therein. The thrombectomy catheter of this and other examples may further include a distal tip member having a length, a lumen extending therein, a central longitudinal axis and a distal opening. The distal tip member may be coupled to the distal end region of the catheter shaft. The distal tip member may further include a spoked wheel disposed within the interior diameter of the distal tip member lumen. The spoked wheel may include a plurality of spokes radially extending from a centrally located hub with an aperture therein; such that the aperture is maintained substantially coaxial with the central longitudinal axis of the distal tip member.

Alternatively or additionally to any of the examples above, the catheter shaft may further include a fluid jet loop disposed therein. The fluid jet loop may include a fluid jet loop lumen with a plurality of jet orifices extending through a wall of the fluid jet loop.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of an example thrombectomy system;

FIG. 2 is a partially exploded perspective view of a portion of the thrombectomy system of FIG. 1;

FIG. 3 is a longitudinal cross-sectional view of a distal end region of an example thrombectomy catheter; and

FIG. 4 is a distal-end view of the distal tip member of an exemplary catheter of the present disclosure.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Thrombectomy catheters, aspiration catheters and systems may be used to remove thrombus, plaques, lesions, clots, etc. from veins or arteries. Some thrombectomy catheters and aspiration catheters may utilize high velocity saline jets in a series to entrain fluid or clot material into and through the shaft of the catheter. Other thrombectomy systems may utilize one or more pressurized saline jets which travel backwards to create a low-pressure zone and a vacuum effect, whereby the vacuum pulls clot material into and through the distal tip and shaft of the catheter. However, prolonged operation of a thrombectomy system may create increased forces placed on the distal tip of the thrombectomy catheter. Accordingly, it may be desirable to design a thrombectomy catheter and/or aspiration catheter which includes a distal tip member coupled near or coupled to the catheter distal end and configured to resist increased forces placed thereon. Thrombectomy systems which include a thrombectomy catheter having a strengthened distal tip configured to resist increased forces placed thereon are disclosed herein.

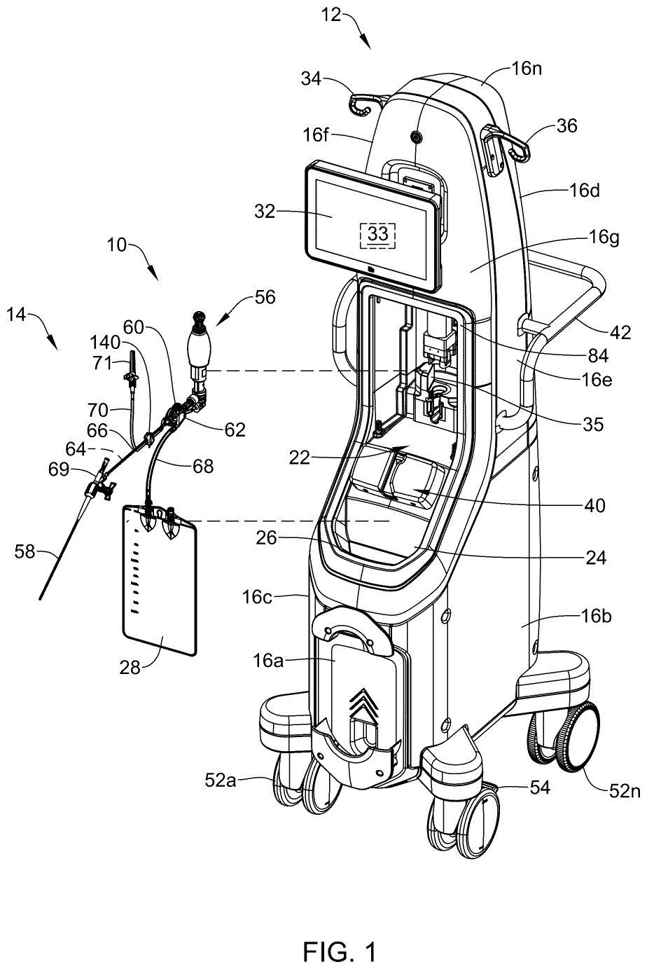

FIG. 1 is a perspective view of an illustrative thrombectomy system 10. The thrombectomy system 10 may include a control console or drive unit 12 and a pump/catheter assembly 14. In some instances, the pump/catheter assembly 14 may be a single use device in which a new pump/catheter assembly 14 may be used with the drive unit 12 for each medical procedure. Shown on the drive unit 12 are a plurality of removable panels 16a-16n about and along the drive unit 12 enclosing the internal structure of the drive unit 12. An illustrative drive unit 12 is described in commonly assigned U.S. Pat. No. 7,935,077, titled THROMBECTOMY CATHETER DEPLOYMENT SYSTEM, the disclosure of which is hereby incorporated by reference. Centrally located in the drive unit 12 and aligned to the lower region of the panel 16g may be automatically opening doors 18 and 20 which open to expose the interior of the drive unit 12 to provide access to a carriage assembly 22. The carriage assembly 22, which may accommodate components of the pump/catheter assembly 14, as discussed further herein, is shown accessible via opening the closed doors 18 and 20. The drive unit 12 may include a catch basin for collecting fluid leakage from the components of the pump/catheter assembly 14. For example, a removable drip tray 24 is shown located on the front of the drive unit 12 extending from below the carriage assembly 22 toward the panel 16a. Other configurations of catch basins are also contemplated. The drip tray 24 and a removable receptacle 26 may collectively support and accommodate an effluent collection bag, such as effluent collection bag 28 of the pump/catheter assembly 14. In other instances, the drive unit 12 may include a different structure, such as a hook for hanging the effluent collection bag 28 from, or a shelf for setting the effluent collection bag 28 on. In instances where the carriage assembly 22 is movable, a carriage assembly activation switch 30 may be provided with the drive unit 12, such as located on panel 16g, to selectively position the carriage assembly 22 inwardly or outwardly. A user interface 32, including memory capabilities, may be provided with the drive unit 12, such as located at the upper region of the drive unit 12 between the upper regions of the upper side panels 16e and 16f. Saline bag hooks 34 and 36 may extend through the panels 16e and 16f to hang saline bags therefrom. The drive unit 12 may include a handle 42 as well as a plurality of wheels 52a-52n and brake pedals 54 for wheel lockage to assist in maneuvering the drive unit 12 by medical personnel.

The pump/catheter assembly 14, which may be a disposable single-use device, is shown unattached from the drive unit 12. The pump/catheter assembly 14 includes a pump 56 and a thrombectomy catheter 58. During use, a portion of the pump/catheter assembly 14 may be secured within a portion of the drive unit 12. Other components included in the pump/catheter assembly 14 may include a bubble trap 60 attached to the pump 56, a connection manifold assembly 62 connected to the bubble trap 60, an effluent return tube 66 connected between the connection manifold assembly 62 and the thrombectomy catheter 58, a high-pressure fluid supply tube 64 attached between the output of the pump 56 and the thrombectomy catheter 58 which may be coaxially arranged inside the effluent return tube 66, a catheter manifold 69 (between the distal end of the effluent return tube 66 and the proximal end of the thrombectomy catheter 58, an effluent waste tube 68 connecting the effluent collection bag 28 to the connection manifold assembly 62, and a fluid supply tube 70 having a bag spike 71 connecting a fluid supply bag 72 (e.g., a saline bag) to the connection manifold assembly 62. The fluid supply tube 70 may be in fluid communication with the interior of the bubble trap 60 to provide fluid from the fluid supply bag 72 to the pump 56 and then to the thrombectomy catheter 58 through the high-pressure fluid supply tube 64.

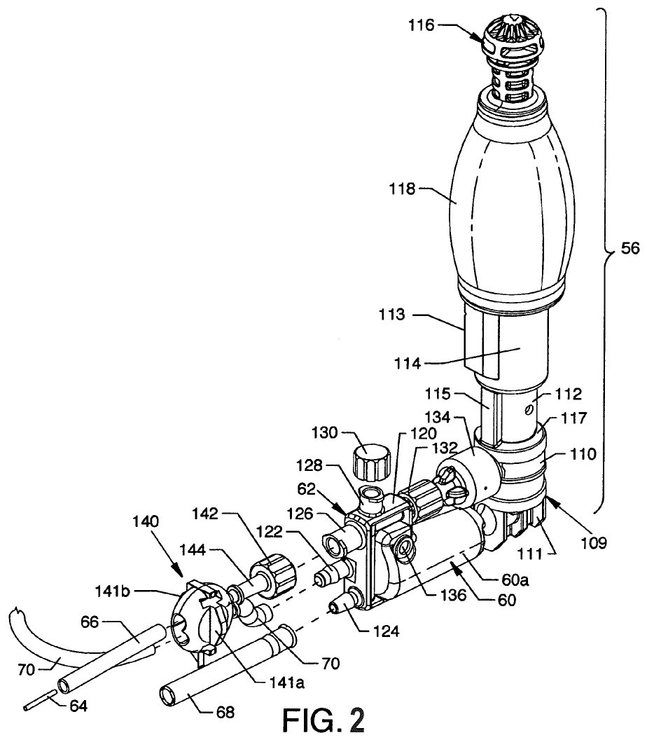

FIG. 2 is a partially exploded perspective view of several components of the pump/catheter assembly 14 generally including the pump 56, the bubble trap 60, the connection manifold assembly 62, and a fixture 140. The pump 56 centers about a tubular body 112. Components are located about the lower region of the tubular body 112 and include a base 109 having an upper portion 110 and a lower portion 111 both positioned about the lower region of the tubular body 112. An annular surface 117 is included at the top of the upper portion 110 of the base 109 for intimate contact with capture tabs of the carriage assembly 22 to contain the pump 56 within the carriage assembly 22. A top body 114, is positioned about the upper region of the tubular body 112. The base 109 and the top body 114, as well as a connecting panel 115, may be molded or otherwise suitably constructed to encompass the greater part of the tubular body 112, for example. A data plate 113 may also be included on the top body 114 for the inclusion of a barcode, an RFID tag, or other informational displays to determine operational parameters of the device.

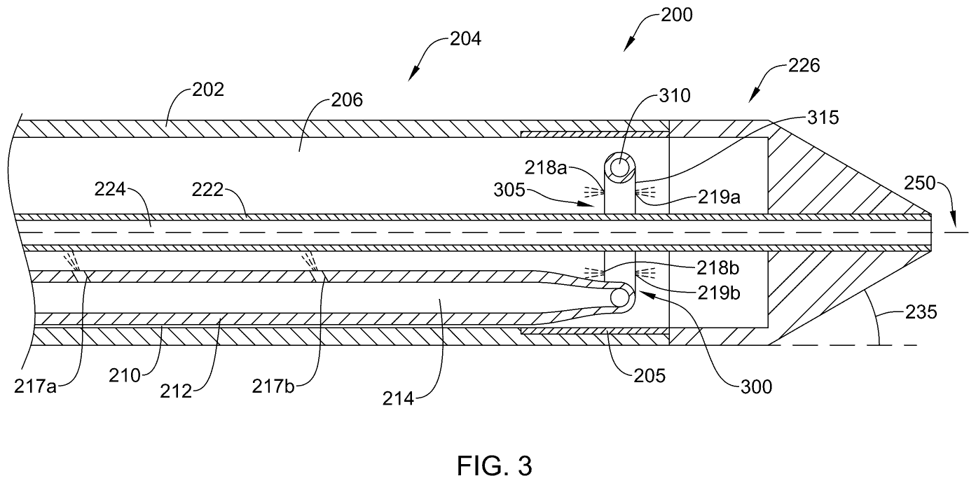

The pump 56 may include a hemispherically-shaped pump piston head 116 having a flexible boot 118 connected to and extending between the top body 114 and the pump piston head 116. In some instances, the geometrically configured lower portion 111 of the base 109 may serve as a mount for one end of the bubble trap 60 (FIG. 3).

The connection manifold assembly 62 may be secured directly to the other end of the bubble trap 60 and in some instances may include a bracket 120 to which is attached a vertically oriented tubular manifold 148 having a plurality of ports attached or formed therethrough including a fluid (e.g., saline) inlet port 122, an effluent outlet port 124, a Luer style effluent return port 126, and/or an auxiliary port 128 and cap 130. Also shown are connectors 132 and 134 connecting extending between the connection manifold assembly 62 and the upper portion 110 of the base 109.

The bubble trap 60 may include mating halves of which one mating half 60a is shown. A hydrophobic filter 136 may be included at the upper forward region of the bubble trap half 60a. Another hydrophobic filter may be included on the second bubble trap half (not explicitly shown) which opposes the hydrophobic filter 136 on the bubble trap half 60a.

The fixture 140, and components associated therewith, assists in support and connection of the effluent return tube 66 to the effluent return port 126 by a connector 142 combined continuously with a connection tube 144, and also assists in support, passage and connection of the fluid supply tube 70 with the fluid inlet port 122. The fixture 140 may include outwardly extending vertically aligned and opposed tabs 141a and 141b which prevent the fixture 140 and associated effluent return tube 66 containing the high-pressure fluid supply tube 64 and the fluid supply tube 70 from contacting a roller pump (not explicitly shown) provided with the drive unit 12, such as located in the carriage assembly 22 or adjacent thereto.

FIG. 3 is a longitudinal cross-sectional view of a distal end region 204 of an illustrative thrombectomy catheter 200. The thrombectomy catheter 200 may be one illustrative example of the thrombectomy catheter 58 described above and further herein may be interchangeably referred to as an aspiration catheter 200. The thrombectomy catheter 200 may include a tubular member or catheter shaft 202 extending from a proximal end region (not explicitly shown) configured to remain outside the patient's body to a distal end region 204. The catheter shaft 202 may be one illustrative example of, or be in fluid communication with, the effluent return tube 66 of the thrombectomy catheter 58 described above. A lumen 206 may extend from the proximal end region to the distal end region 204 of the catheter shaft 202. While not explicitly shown, the catheter shaft 202 may include one or more markers (e.g., radiopaque marker bands) disposed along the catheter shaft 202. Further, while not explicitly shown, in some embodiments, the catheter shaft 202 may include one or more openings extending through a sidewall thereof, if desired.

It can be appreciated that the catheter shaft 202 may be formed from a polymer material. For example, the catheter shaft 202 may be formed from a polymer material including, but not limited to a thermoplastic polymer (e.g., Pebax®). Other suitable polymers which may be utilized to form the tip member 226 may include Vestamid®, Grilamid®, polyamides including Nylon 6, Nylon 66, Nylon 11, Nylon 12, polyether block amide copolymer including 32D Pebax®, 35D Pebax®, 48D Pebax®, 55D Pebax®, 68D Pebax®, 72D Pebax®, Pebax® MED, Rilsan® MED, Rilsamid® MED, Rilsan® Clear MED and Kynar® MED. A non-limiting list of examples which may be utilized to form the catheter shaft 202 is disclosed below.

The thrombectomy catheter 200 may further include a distal tip member 226 positioned along the distal end region 204. For instance, the distal tip member 226 may be fixed or secured to a distal end of the catheter shaft 202 and extend distally therefrom. The distal tip member 226 may be tapered along at least a portion of the distal tip member 226. In this and other examples, the distal tip member 226 may be tapered distally to a smaller diameter along at least a portion of the distal tip member 226 by an angle of at least 15 degrees with respect to the central longitudinal axis 250 of the distal tip member 226. In other words, the tapering angle for at least a portion of the distal tip member 226 may ascribe to an angle of at least 15 degrees or more with respect to the central longitudinal axis 250 of the catheter 200. Other angles and ranges of angles are contemplated. Alternatively or additionally, the distal tip member 226 may be tapered over its entire structure, substantially over its entire structure, or intermittently over its entire structure, a part of its structure, or portions of its structure, and at a tapering angle with respect to the central longitudinal axis 250 of the distal tip member 226. In this and other examples, the tapering angle with respect to the central longitudinal axis 250 of the distal tip member 226 may range from 5 to 85 degrees. In other non-limiting examples, the tapering angle may range from 10 to 30 degrees. In yet other non-limiting examples, the tapering angle may range from 25 to 60 degrees. The distal tip member 226 may be formed of a polymeric material, such as those materials listed above with regard to the catheter shaft 202. In some instances, the distal tip member 226 may be formed of a polymeric material having a durometer hardness less than the polymeric material of the catheter shaft 202 to provide an atraumatic distal tip.

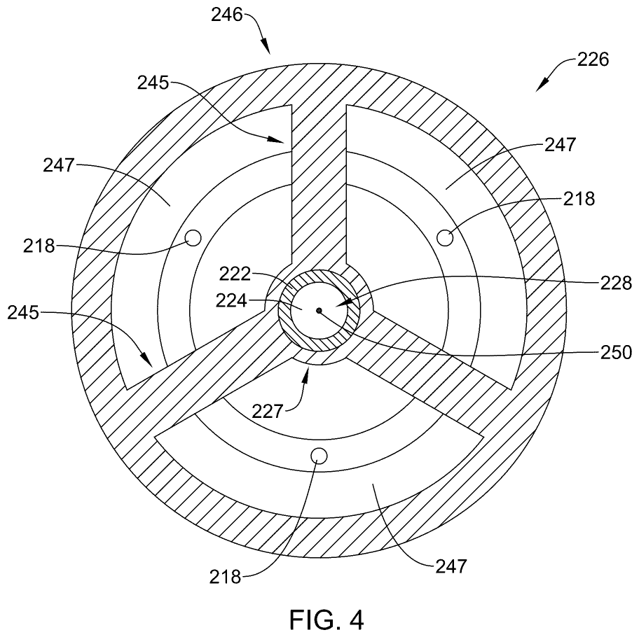

FIG. 4 shows a distal-end cross-sectional view of the distal tip member 226 of FIG. 3. Distal tip member 226 may further include a plurality of spokes 245 extending between the guidewire shaft 222 and an inner surface of the distal tip member 226. As shown in FIG. 4, distal tip member 226 may further include a hub 227 with an aperture 228 disposed centrally within the hub 227. In some instances, the hub 227 may be coaxial with the central longitudinal axis 250. The spokes 245 may extend radially outward from the hub 227. The hub 227 and the plurality of spokes 245 in combination may be considered a spoked wheel 246. The plurality of spokes 245 may define a plurality of passages 247 between each of the spokes 245 of the plurality of spokes 245. In other words, a passage 247 may be defined between a circumferentially adjacent spokes 245. The plurality of passages 247 are configured such that entrained fluid may pass through the plurality of passages 247 from distal of the distal tip member 226 and into the lumen 206 of the catheter shaft 202.

The hub 227 may be further configured to accept a guidewire shaft 222 into or through aperture 228 of the hub, with a guidewire lumen 224 of the guidewire shaft 222 disposed coaxially within the hub 227. The hub 227, in coordination with spokes 245, acts to maintain the guidewire shaft 222, and therefore the guidewire lumen 224, substantially coaxial with the central longitudinal axis 250 of the distal tip member 226. In other words, the hub 227 and spokes 245 not only reinforce structural integrity within the catheter shaft 202, but also hold the guidewire shaft 222 substantially in place within the center of the catheter shaft lumen 206. This feature is advantageous as it improves the traversal of a medical device through tortuous internal anatomy by keeping the guidewire shaft 222 substantially centered within the catheter 200 (or other medical device), thereby reducing the chances of a guidewire and/or the distal tip member 226 making unwanted contact with vessel walls or lumen walls or other related structures. By maintaining the guidewire shaft 222, and therefore the guidewire lumen 224 substantially centered, the distal tip member 226 is also less subject to prolapse, in which the distal tip member 226 may bend or deform upon contact with the surface of a vessel or lumen wall or upon contact with an obstruction. Another advantage of the innovative constructions disclosed is increased structural integrity provided to the distal tip member 226 by the hub 227 and spokes 245. Additionally, the hub 227 and spokes 245 of the distal tip member 226 may increase the applied surface area of the distal tip member 226. In other words, the hub 227 and spokes 245 of the distal tip member 226 may increase the amount of surface area available for treatment at the treatment site, as the hub 227 and spokes 245 reduce and/or prevent prolapse or other deformations of the distal tip member 226, thereby affording a greater amount of surface area available for treatment, such as in a thrombectomy procedure, which improves the rapidity and effectiveness of thrombus removal and the removal of other obstructions. Further, the coordination of hub 227, aperture 228, and spokes 245 of the spoked wheel 246 may help maintain the guidewire shaft 222, and therefore the guidewire lumen 224, centered within the catheter shaft lumen 206 and the lumen of the distal tip member 226. This construction provides greater flexibility of the catheter 200 while improving the trackability of the catheter 200 through tortuous internal anatomy such as vessels, lumens, and the like.

Alternatively or additionally to any of the examples disclosed herein, the spoked wheel 246 may be molded as a single component and installed within the distal tip member 226. In this and other examples, the spoked wheel 246 may include a hub 227 centrally located therein. The hub 227 may further include an aperture 228 which is configured to accept the passage of any feasible medical device with an external diameter less than the diameter of the aperture 228. In this and other examples, the hub 227 and aperture 228 may accept passage of the guidewire shaft 222 defining the guidewire lumen 224. In this and other examples, the spoked wheel 246 may include at least one spoke 245. In non-limiting examples, the spoked wheel 246 may include a plurality of spokes 245, and more than one passage 247 may be formed between the spokes 245 which allow for the passage of high pressure fluid and/or other media (e.g., jetted motive fluid, thrombi, clot material, bodily fluid, bodily tissue, bodily obstructions, etc.) to pass through the passages 247. In this and other examples, the passages 247 may be arcuate, circular, triangular, wedge-shaped, or conform to any shape or geometry desired. In yet other non-limiting examples, the spoked wheel 246 may include at least two spokes, at least three spokes, at least four spokes, at least five spokes, or at least six or more spokes.

Alternatively or additionally to the examples provided herein, the distal tip member 226 and its associated components (i.e., hub 227, aperture 228, spokes 245, spoked wheel 246, etc.) may be formed by a multi-lumen extrusion process or by a multi-lumen extrusion process which further incorporates a post-extrusion shaping process. In other non-limiting examples, the distal tip member 226 and its associated components may be molded as a one-piece construction, a two-piece construction, a three-piece construction, a four-piece construction, or a construction of five or more pieces or components. In other non-limiting examples, the distal tip member 226 and its associated components may be attached to the catheter shaft 202 by myriad processes, including but not limited to reflow processes, adhesive processes, mechanical bonding processes (i.e., threaded connection between components, snap-fit connection between components, etc.), overmold processes, or the like.

FIG. 3 further the guidewire shaft 222 may extend within the lumen 206 of the catheter shaft 202. In some examples, a proximal end of the guidewire shaft 222 may be coupled (e.g., attached, bonded) to the catheter manifold 69 (shown in FIG. 1). Further, FIG. 3 illustrates that the guidewire shaft 222 may include a lumen 224 configured to permit a guidewire to extend within.

The guidewire shaft 222 may be formed from a polymer material including, but not limited to a polyimide. Other suitable polymers which may be utilized to form the guidewire shaft 222 may include PEEK (polyether ether ketone). A non-limiting list of examples which may be utilized to form the guidewire shaft 222 is disclosed below.

It can be appreciated that constructing the guidewire shaft 222 from a thermoset polyimide provides sufficient strength to the guidewire shaft 222, thereby allowing the guidewire shaft 222 to withstand forces of the jetted motive fluid leaving the jet orifices 217 of the high-pressure fluid supply tube 210, described herein. However, it can be further appreciated that the thermoset polyimide material utilized to form the guidewire shaft 222 may not be capable of forming a chemical bond to the thermoplastic polymer used to form the catheter shaft 202 and the tip member 226. Accordingly, it may be desirable to design a portion of the guidewire shaft 222 to include one or more features which permit the thermoset polyimide material utilized to form the guidewire shaft 222 to form a mechanical bond to the thermoplastic polymer used to form the distal tip member 226.

The guidewire shaft 222 may accommodate a guidewire lumen 224 therein, and may be disposed concentrically within catheter 200. In other words, the guidewire shaft 222 may be located coaxially with the catheter shaft 202 along the central longitudinal axis 250 of catheter 200, or at least substantially coaxially with the catheter shaft 202 along the central longitudinal axis 250. The securement of the guidewire shaft 222 to the distal tip member 226 (i.e., securement of the guidewire shaft 22 within the hub 227 of the distal tip member 226 may position the guidewire shaft 222 coaxial with the catheter shaft 202 along the central longitudinal axis 250. Alternatively or additionally, the guidewire shaft 222 may be located within, but not coaxial with the catheter shaft 202. For instance, in other non-limiting examples, the guidewire shaft 222 may be attached to or near an interior wall of the catheter shaft 202.

The catheter shaft 202 of the catheter 200 may further include a saddle region 205 or equivalent reinforcing member. Saddle region 205 may be designed to add structural integrity to the catheter shaft 202 of catheter 200 such that the catheter 200 may resist deformation and damage under high fluid forces. The saddle region 205 may be formed partially or entirely of stainless steel, or may be formed partially or entirely by materials including but not limited to: an alloy, a steel, iron, a metal, a plastic, a thermoplastic, a galvanized alloy, a galvanized metal, or the like.

The thrombectomy catheter/aspiration catheter 200 may further include a high-pressure fluid supply tube 210. The high-pressure fluid supply tube 210 may be one illustrative example of, or be in fluid communication with, the high-pressure fluid supply tube 66 of the thrombectomy catheter 58 described above. The high-pressure fluid supply tube 210 may be disposed within and extend through the lumen 206 of the catheter shaft 202. The high-pressure fluid supply tube 210 may include a supply tube wall 212 defining a lumen or fluid pathway 214 extending therethrough. In at least some instances, the high-pressure fluid supply tube 210 may include a fluid jet loop 300 provided at a distal end thereof. In other instances, the high-pressure fluid supply tube 210 may have a closed distal end preventing fluid from exiting the distal end. The high-pressure fluid supply tube 210 may extend along a length of the catheter shaft 202 with the distal end (e.g., the fluid jet loop 300 located within the lumen 206 of the catheter shaft 202 proximal to the distal tip member 226 secured at the distal end of the catheter shaft 202. In other instances, the distal end (e.g., the fluid jet loop 300) of the high-pressure fluid supply tube 210 may extend into a proximal portion of the distal tip member 226. A proximal end of the high-pressure fluid supply tube 210 may be in fluid communication with the pump 56 described herein, to provide high-pressure fluid to the fluid pathway 214 of the high-pressure fluid supply tube 210.

A plurality of jet orifices 217a-b (collectively, 217) may be defined along the supply tube wall 212. For example, the supply tube wall 212 may include two, three, four, five, six, or more jet orifices 217. The jet orifices 217 may be spaced along the supply tube wall 212 at any desired intervals. For example, each of the jet orifices 217 may be equidistantly spaced from adjacent jet orifices 217 along the length of the high-pressure fluid supply tube 210, i.e., along the length of the supply tube wall 212. In other instances, the jet orifices 217 may be arranged such that the spacing between adjacent jet orifices 217 near the distal end of the supply tube wall 212 is closer than the spacing between adjacent jet orifices 217 near the proximal end of the supply tube wall 212. For instance, the spacing between the orifices 217 may gradually increase as you move proximally along the length of the shaft, or the spacing may increase in a stepwise configuration. In some instances, some or all of the jet orifices 217 may be axially aligned along the supply tube wall 212. In other instances, one or more of the jet orifices 217 may be circumferentially offset from one another about the supply tube wall 212. A number of patterns are contemplated including a helical pattern, a pattern where no two jet orifices 217 are disposed at the same axial location, a regular pattern including two or more jet orifices 217 disposed at the same axial location, an irregular pattern (where some of the jet orifices 217 may or may not be disposed at the same axial location), etc.

The jet orifices 217 may be formed using a suitable method such as electron discharge machining, etching, cutting (e.g., including laser cutting), or the like. In some instances, one or more of the jet orifices 217 may have a substantially round shape. In other instances, one or more of the jet orifices 217 may have a substantially non-round shape (e.g., oval, polygonal, irregular, etc.). In some instances, the jet orifices 217 may be beveled or otherwise include a beveled surface. It is contemplated that a size and/or a shape of the jet orifices 217 may be varied to vary the velocity of the fluid exiting the jet orifices. For example, decreasing the size of the jet orifices 217 may increase the velocity of the fluid exiting the jet orifices 217. In some embodiments, the size of the jet orifices 217 may be varied based on the pressure capacity of the thrombectomy system, the number of jet orifices, the dimensions of the high-pressure fluid supply tube 210 (e.g., length, wall thickness, inner diameter, etc.), and/or combinations thereof. In some examples, the jet orifices 217 may have a cross-sectional dimension in the range of about 0.0018″ (0.0018 inches) to about 0.0022″. However, the jet orifices 217 can have a cross-sectional dimension of less than 0.0018″ or greater than 0.0022″, as desired.

Infusion of motive fluid through the lumen 214 of the supply tube wall 212 may result in fluid being jetted through the jet orifices 217 and the generation of a proximally directed aspiration force. At least some of the jet orifices (217a, 217b, etc.) may be angled in a proximal direction or otherwise designed to infuse fluid (e.g., a motive fluid, a liquid, a gas or air, steam, a fluid with particles disposed therein, or the like) through the jet orifices 217 and into the lumen 206 of the catheter shaft 202 in a generally proximal direction as depicted in FIG. 3, representing motive jetted fluid projecting generally proximally from the jet orifices 217a and 217b. For example, each of the jet orifices (217a, 217b, etc.) may be arranged at an acute angle to the longitudinal axis of the supply tube wall 212 such that the jet orifices 217 angle in a proximal direction.

In some embodiments, one or more of the jet orifices (not shown) along the length of the supply tube wall 212 may be designed to infuse fluid (e.g., a motive fluid, a liquid, a gas or air, steam, a fluid with particles disposed therein, or the like) through the jet orifice(s) 217 and into the lumen 206 of the catheter shaft 202 in a generally distal direction from the jet orifice. For example, one or more of the jet orifices may be arranged at an oblique angle to the longitudinal axis of the supply tube wall 212 such that one or more jet orifices angles in a distal direction. It is contemplated that an angle of the jet orifices and thus the motive jetted fluid may be varied to adjust the velocity of the fluid exiting the jet orifices.

In some embodiments, the supply tube wall 212 may include one or more, or a plurality of proximally oriented or directed jet orifices 217a, 217b, etc. (i.e., jet orifices configured to direct fluid infused through the lumen 214 of the supply tube wall 212 in a proximal direction), and the supply tube wall 212 may include one or more, or a plurality of distally oriented or directed jet orifices (i.e., jet orifices configured to direct fluid infused through the lumen 214 of the supply tube wall 212 in a distal direction). In some examples, the distally projecting jet orifices may be axially aligned with one or more of the proximally projecting jet orifices 217. In other examples, one or more distally projecting jet orifices may be circumferentially offset from one or more of the proximally projecting jet orifices 217. For example, one or more distally projecting jet orifices may be circumferentially offset from one or more of the proximally projecting jet orifices 217 in the range of about 10° to about 350° or about 45° to about 135°.

A distally projecting jet orifice may be the distalmost jet orifice, with the proximally projecting jet orifices 217 positioned proximal of the distally projecting jet orifice 217. However, this is not required. In some embodiments, one or more distally projecting jet orifices may be positioned proximal to at least one proximally projecting jet orifice 217. The supply tube wall 212 may include more than one distally projecting jet orifice, as desired. When more than one distally projecting jet orifice is provided, the distally projecting jet orifices may be positioned at differing axial and/or circumferential locations from one another or similar axial and/or circumferential locations as one another, as desired. The distally projecting jet orifice(s) may break up particles as they are drawn into the lumen 206 of the catheter shaft 202 while the proximally projecting jet orifices 217 may move particles proximally along the catheter shaft 202.

The performance of the thrombectomy catheter 200 and the high-pressure fluid supply tube 210 may be directly related to the velocity of the motive jetted fluid exiting the jet orifices 217 and the shear-induced turbulent flux created by the jetted motive fluid. For example, the more powerful the jetted motive fluid, the higher the aspiration rates may be. It is further contemplated that the performance of the jet-powered aspiration catheter 200 may be directly related to the speed at which the clot can be entrained into the catheter 200, macerated, and removed from the shaft. Any clogging that occurs within the catheter shaft 202 may reduce or completely stop the removal of the clot. The addition of a distally projecting jet orifice may macerate any clot that enters the distal opening 208 of the catheter shaft 202 thus helping prevent clogging. For example, at the point of impingement of the distally oriented motive jetted fluid, the motive jetted fluid may deflect distally creating flow distally out the passages 247 of the distal tip member 226, effectively macerating any clot that enters the tip of the device and eliminating or reducing risk of the passages 247 of the distal tip member 226 becoming blocked or clogged. It is contemplated that the properties (size, shape, angle, number, spacing, etc.) of the jet orifices 217 may be varied to obtain a fluid velocity that creates an optimum de-clogging effect without hindering the proximal flow of a clot within the lumen 206 of the catheter shaft 202 or the clot evacuation rate.

In some instances, the jet orifices 217 may be oriented at an oblique angle relative to the longitudinal axis of the supply tube wall 212. For example, the proximally projecting jet orifices 217a-b may be oriented at an oblique (e.g., acute) angle relative to the longitudinal axis of the supply tube wall 212 and/or oriented at an angle greater than zero degrees and less than ninety degrees relative to the longitudinal axis of the supply tube wall 212. It is contemplated that a distally projecting jet orifice, if present, may be oriented at an oblique (e.g., obtuse) angle relative to the longitudinal axis of the supply tube wall 212 and/or oriented at an angle greater than 90 degrees and less than 180 degrees relative to the longitudinal axis of the supply tube wall 212. In other instances, the jet orifices 217 may be oriented perpendicular to the longitudinal axis of the supply tube wall 212 (e.g., at an angle of about 90 degrees relative to the longitudinal axis of the supply tube wall 212). The angle may or may not be the same for all the jet orifices 217.

In at least some instances, the jet orifices 217 may be understood as being arranged in series. In other words, the jet orifices 217 may be arranged such that adjacent jet orifices 217 are spaced longitudinally apart at various locations along the longitudinal axis of the supply tube wall 212. For example, the jet orifices 217 may be uniformly or non-uniformly spaced along a length of the supply tube wall 212. This may position the jet orifices 217 at axially spaced apart locations within the catheter shaft 202 and along the length thereof. For example, the jet orifices 217 may be spaced along an entire length of the supply tube wall 212 and correspondingly along an entire length of the catheter shaft 202, or portions thereof, as desired. In some examples, the jet orifices 217 may be spaced at intervals in the range of every 5 inches to every 15, or in the range of every 6 inches to every 12 inches along a length of the supply tube wall 212. In other instances, the spacing between the jet orifices 217 may be less than every 5 inches or greater than every 15 inches. Accordingly, motive fluid leaves via the jet orifices 217 forming a jetted motive fluid. This jetted motive fluid enters an entrainment material where the shear layer between the two causes turbulence, mixing, and transfer of momentum. Entrainment material may enter the passages 247 through the distal tip member 226 and then may be urged proximally by momentum transfer. As the mixture of jetted motive fluid and entrainment material migrates proximally, the material may sequentially approach a number of jet orifices 217. Upon interaction with the jetted motive fluid from each individual jet orifice 217, the momentum in the entrainment material mixture may increase, and the thrombogenic material may more readily flow proximally through the catheter shaft 202 for removal. The increase in momentum may allow for the catheter shaft 202 to be used without a second or outflow orifice (e.g., positioned proximally of the distal tip member 226). Alternatively, some of the entrapped thrombogenic material may exit the catheter shaft 202 through a second orifice (not shown), e.g., in a sidewall of the catheter shaft 202, positioned proximal to the distal tip member 226, recirculate to the passages 247 of the distal tip member 226 (e.g., one or more times), and then move proximally through the lumen 206 of the catheter shaft 202.

The thrombectomy/aspiration catheter 200 of this and other examples may further include a fluid jet loop 300 formed at the distal end of the high-pressure fluid supply tube 210. FIG. 3 depicts an example of a fluid jet loop 300 as contemplated by the present disclosure. The fluid jet loop 300 may include a jet loop lumen 310 extending therein and a plurality of jet orifices 218a-b which may face proximally (collectively 218) and/or a plurality of jet orifices 219a-b which may face distally (collectively 219), each extending through a wall 315 of the fluid jet loop 300. For example, each of the proximally facing jet orifices 218 may be arranged to infuse fluid in a proximal direction, e.g., parallel to or at an acute angle to the longitudinal axis of the supply tube wall 212 such that the jet orifices 218 are oriented in a proximal direction. Additionally, each of the distally facing jet orifices 219 may be arranged to infuse fluid in a distal direction, e.g., parallel to or at an acute angle to the longitudinal axis of the supply tube wall 212 such that the jet orifices 219 are oriented in a distal direction. In some embodiments, one or more of the jet orifices 218 and 219 may be designed to infuse fluid (e.g., a motive fluid, a liquid, a gas or air, steam, a fluid with particles disposed therein, or the like) through the jet orifice(s) 218 and 219 from the high-pressure fluid supply tube 210 into the lumen 206 of the catheter shaft 202. In some instances, the jet orifices 218 may be arranged parallel to or at oblique angles to the central longitudinal axis 250 such that the jet orifices 218 are oriented in a proximal direction. Additionally, the jet orifices 219 may be arranged parallel to or at oblique angles to the central longitudinal axis 250 such that the jet orifices 219 are oriented in a distal direction. It is contemplated that an angle of the jet orifices 218/219 and thus the motive jetted fluid may be varied to adjust the velocity of the fluid exiting the jet orifices 218/219.

The fluid jet loop 300 may be positionable within the catheter shaft lumen 206 and may be further positionable such that at least a portion of the fluid jet loop 300 encircles at least a portion of the guidewire shaft 222. In this and other examples, the distal end or distal end portion of the fluid jet loop 300 may be positionable within the catheter shaft lumen 206 such that at least a portion of the fluid jet loop 300 encircles at least a portion of the guidewire lumen 224, such that the plurality of proximally facing or directed jet orifices 218 are oriented in a proximal direction and/or such that the plurality of distally facing or directed jet orifices 219 are oriented in a distal direction. In some instances, the guidewire shaft 222 may extend through the fluid jet loop 300 such that the fluid jet loop 300 circumferentially surrounds the guidewire shaft 222.

One or more of the plurality of jet orifices 218 and/or 219 of the fluid jet loop 300 may be oblong, circular, substantially circular, ellipsoidal, rectangular, square, v-shaped, u-shaped, z-shaped, s-shaped, w-shaped, tear-drop-shaped, pear-shaped, slit-shaped, rhomboid, or conform to any equivalent geometric shape as desired.

Alternatively or additionally to any of the examples disclosed herein, the fluid jet loop 300 may be positionable within the body of catheter 200 in an orientation that directs the fluid jet orifices 218 and 219 in the desired direction. In this and other examples, the desired direction may be a proximal direction with respect to the orientation of the catheter 200. In other non-limiting examples, the desired direction may be a distal direction, a lateral direction, or a radial direction with respect to the orientation of the catheter 200.

The proximal end of fluid jet loop 300 may be connected to or be formed as the high-pressure fluid supply tube 210 or equivalent structure which supplies fluid (e.g., saline, jetted fluid) or other media at high velocity and high pressure to each of the fluid jet orifices 218 and/or 219.

The fluid jet loop 300 may include a toroidal region 305 at or near its distal end. In other words, the fluid jet loop 300 may curve to form a toroidal or torus shape at its distal end. In this and other examples, the toroidal region 305 of the fluid jet loop 300 may be arranged such that the plurality of jet orifices 218 are oriented in a proximal direction, allowing high velocity and high-pressure fluid to move back through the catheter lumen 206 and therefore create a low-pressure zone via the Bernoulli effect, allowing thrombi and other obstructions to be aspirated into aspiration/thrombectomy catheter 200 through the passages 247 of the distal tip member 226 by a relative vacuum created by a large difference in pressures supplied by the aspiration catheter 200 to the treatment site and desired areas of treatment. In other non-limiting examples, the toroidal region 305 of the fluid jet loop 300 may be arranged such that the plurality of jet orifices 219 are oriented in a distal direction, allowing high velocity and high-pressure fluid to be expelled distally out of the passages 247 of the distal tip member 226 between the spokes 245.

The toroidal region 305 may be placed proximate the spoked wheel 246 of the distal tip member 226. In this and other non-limiting examples, the toroidal region 305 may be placed proximate of the spoked wheel 246 (which includes spokes 245, hub 227 and aperture 228) by a distance of about 2 mm. In other non-limiting examples, the toroidal region 305 may be placed proximate of the spoked wheel 246 by a distance of about 3 mm. In yet other non-limiting examples, the toroidal region 305 may be placed proximate of the spoked wheel 246 by a distance of about 0.25 mm, about 0.5 mm, about 0.75 mm, about 1 mm, about 1.25 mm, about 1.5 mm, about 1.75 mm, about 2.25 mm, about 2.5 mm, about 2.75 mm, about 3.25 mm, about 3.5 mm, or more.

The toroidal region 305 of the fluid jet loop 300 may be designed such that the toroidal region 305 encircles the guidewire shaft 222. In other non-limiting examples, the toroidal region 305 of the fluid jet loop 300 may be alternatively designed as ascribing to a helical shape as it encircles the guidewire shaft 222. In other non-limiting examples, the toroidal region 305 of the fluid jet loop 300 may be alternatively designed as or take the form of a halo shape as it encircles the guidewire shaft 222. In yet other non-limiting examples, the toroidal region 305 can be alternatively designed to ascribe to myriad geometric patterns as it encircles the guidewire shaft 222.

Multiple interior diameter cross-sections of the lumen 310 of the fluid jet loop 300 are contemplated. In non-limiting examples, the interior diameter cross-section of the lumen 310 of the fluid jet loop 300 may be fully or substantially triangular. In other non-limiting examples, the interior diameter cross-section of the lumen 310 of the fluid jet loop 300 may be fully or substantially circular, fully or substantially oblong, fully or substantially oval, fully or substantially square, fully or substantially rectangular, or other desired shape It is further contemplated that fluid jet loop 300 may provide an interior diameter cross-section that changes shape along its length and/or changes its shape intermittently along its length. In other words, a portion of the fluid jet loop 300 may have a different interior diameter cross-sectional shape and diameter than a portion of the fluid jet loop remote from that portion. In non-limiting instances, the interior diameter cross-sectional shape of the fluid jet loop 300 may change multiple times along its length, or may change only once along its length, however, myriad combinations of the aforementioned cross-sectional shapes, patterns, and implementations may be utilized as desired.

The fluid jet loop 300 of this and other examples may be constructed such that the fluid jet loop orifices 219 are oriented in a distal direction and may direct the passage of high-pressure fluid or other media through the passages 247 defined by the spaces between spokes 245 and hub 227 of the spoked wheel 246. In other words, the toroidal region 305 or at least a portion of the fluid jet loop 300 may encircle the guidewire shaft 222 such that the fluid jet loop orifices 219 are aligned with the passages 247 and oriented in a distal direction to direct the passage of high-pressure fluid or other media through the passages 247 defined by the spaces between spokes 245 and hub 227 of the spoked wheel 246. In other words, the toroidal region 305 or at least a portion of the fluid jet loop 300 may encircle the guidewire shaft 222 such that the fluid jet loop orifices 219 are oriented in a distal direction and may direct the passage of high-pressure fluid or other media through the passages 247 defined by the spaces between spokes 245 and hub 227 of the spoked wheel 246. In this and other examples, the toroidal region 305 may confine or ascribe to a geometric pattern and/or shape including but not limited to a toroid, a torus shape, a circular pattern, a circular shape, a halo pattern, a halo shape, a helical pattern, a helical shape, or any other configuration desire.

The materials that can be used for the various components of the catheter 200 may include those commonly associated with medical devices. However, this is not intended to limit the devices and methods described herein, as the discussion may be applied to other components, devices, or systems disclosed herein.

The components of the catheter 200 (and/or other systems disclosed herein) may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the components of the catheter 200 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the components of the catheter 200 (and/or other systems disclosed herein) in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the components of the system 10 (and/or other systems disclosed herein) to achieve the same result.

In some embodiments, a degree of Magnetic Resonance Imaging (MRI) compatibility is imparted into the catheter 200 (and/or other systems disclosed herein). For example, components of the catheter 200 (and/or other systems disclosed herein), may be made of a material that does not substantially distort the image and create substantial artifacts (e.g., gaps in the image). Certain ferromagnetic materials, for example, may not be suitable because they may create artifacts in an MRI image. The components of the catheter 200 (and/or other systems disclosed herein) or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Alternatively or additionally to any of the examples disclosed herein, and although not explicitly shown, a radiopaque marker band may be disposed about the outer circumferential surface of the catheter shaft 202 and located near the distal tip member 226 or near the catheter 200 distal end region 204. One or more radiopaque marker bands may be present and may alternatively take the form of a radiopaque coating, a radiopaque section, a strip of radiopaque material, a strip of radiopaque material comprising indicia and/or a pattern (i.e., a helical pattern, a zig-zag pattern, a serpentine pattern, an undulating pattern, a sinusoidal pattern, or the like).

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An aspiration catheter, comprising:
a catheter shaft having a proximal end region, a distal end region and a lumen extending therein;
a distal tip member coupled to the distal end region of the catheter shaft;
a guidewire shaft having a proximal end region, a distal end region and a guidewire lumen extending therein;
wherein the guidewire shaft extends within at least a portion of the lumen of the catheter shaft and within the distal tip member;
wherein the distal tip member includes a plurality of spokes extending between the guidewire shaft and an inner surface of the distal tip member.

2. The aspiration catheter of claim 1, wherein the distal tip member includes a hub with an aperture therein, wherein the guidewire shaft is configured to extend through the aperture of the hub.

3. The aspiration catheter of claim 2, wherein the aperture of the hub is centrally located along a central longitudinal axis of the distal tip member, such that the plurality of spokes maintain the guidewire lumen substantially coaxial with the central longitudinal axis of the distal tip member.

4. The aspiration catheter of claim 1, wherein the plurality of spokes define a plurality of passages between the plurality of spokes, the plurality of passages configured to allow passage of entrained fluid into the lumen of the catheter shaft.

5. The aspiration catheter of claim 1, wherein a distal portion of the distal tip member is tapered at an angle of at least 15 degrees with respect to a central longitudinal axis of the distal tip member.

6. The aspiration catheter of claim 1, wherein the catheter shaft further includes a fluid jet loop disposed therein, the fluid jet loop including a fluid jet loop lumen with a plurality of jet orifices extending through a wall of the fluid jet loop.

7. The aspiration catheter of claim 6, wherein the fluid jet loop encircles at least a portion of the guidewire shaft such that the plurality of jet orifices are oriented in a proximal direction.

8. The aspiration catheter of claim 6, wherein the fluid jet loop includes one or more distally facing jet orifices oriented toward the distal tip member.

9. The aspiration catheter of claim 6, wherein the one or more distally facing jet orifices are directed toward the plurality of passages of the distal tip member.

10. An aspiration catheter, comprising:
a catheter shaft having a proximal end region, a distal end region, a lumen extending therein, and a central longitudinal axis;
a distal tip member coupled to the distal end region of the catheter shaft;
a guidewire shaft having a proximal end region, a distal end region and a guidewire lumen extending therein;
wherein the guidewire shaft extends within at least a portion of the lumen of the catheter shaft and within the distal tip member;
wherein the distal tip member includes a plurality of spokes radially arranged around the guidewire shaft to support the guidewire shaft such that the guidewire shaft is coaxial with the central longitudinal axis.

11. The aspiration catheter of claim 10, wherein the plurality of spokes define a plurality of passages therebetween, the plurality of passages configured to allow passage of entrained fluid into the lumen of the catheter shaft.

12. The aspiration catheter of claim 10, wherein a distal portion of the distal tip member is tapered distally toward the central longitudinal axis at an angle of at least 15 degrees with respect to the central longitudinal axis.

13. The aspiration catheter of claim 10, wherein the catheter shaft further includes a fluid jet loop disposed therein, the fluid jet loop including a fluid jet loop lumen with a plurality of jet orifices extending through a wall of the fluid jet loop, and wherein the fluid jet loop encircles the guidewire shaft.

14. The aspiration catheter of claim 13, wherein the fluid jet loop includes one or more distally facing jet orifices oriented toward the distal tip member.

15. A thrombectomy catheter, comprising:
a catheter shaft having a proximal end region, a distal end region and a lumen extending therein;
a distal tip member having a length, a lumen extending therein, a central longitudinal axis and a plurality of distal openings;
wherein the distal tip member is coupled to the distal end region of the catheter shaft;
wherein the distal tip member comprises a plurality of radially extending spokes defining the plurality of distal openings therebetween;
a guidewire shaft defining a guidewire lumen;
wherein the guidewire shaft is maintained substantially coaxial with the central longitudinal axis of the distal tip member; and
wherein the plurality of distal openings are arranged radially around the guidewire shaft.

16. The thrombectomy catheter of claim 15, wherein the guidewire shaft extends distally into the distal tip member.

17. The thrombectomy catheter of claim 16, wherein distal tip member includes a central hub, wherein the guidewire shaft is configured to extend into an aperture of the central hub.

18. The thrombectomy catheter of claim 15, wherein a distal portion of the distal tip member is tapered distally toward the central longitudinal axis at an angle of at least 15 degrees with respect to the central longitudinal axis of the distal tip member.

19. The thrombectomy catheter of claim 15, wherein the catheter shaft further includes a fluid jet loop disposed therein, the fluid jet loop including a fluid jet loop lumen with a plurality of jet orifices extending through a wall of the fluid jet loop.

20. The thrombectomy catheter of claim 19, wherein the fluid jet loop encircles at least a portion of the guidewire shaft such that the plurality of jet orifices are oriented in a proximal direction.
